(19) United States
(12) Patent Application Publication
(10) Pub. No.: US 2002/0081283 A1
(43) Pub. Date: Jun. 27, 2002

(54) Reversibly immortalized hepatocytes and methods of use

(76) Inventors: Ira J. Fox, Omaha, NE (US); Philippe LcBoulch, Charleston, MA (US); Naoya Kobayashi, Okayama (JP)

Correspondence Address: SAUL EWING, LLP, Centre Square West, 38th Floor, 1500 Market Street, Philadelphia, PA, 19102-2186, US

(21) Appl. No.: 09/816,785

(22) Filed: Mar. 22, 2001

Related U.S. Application Data
(60) Provisional application No. 60/101,812, filed on Sep. 25, 1998.

(30) Foreign Application Priority Data
Sep. 24, 1999 (US) PCT/US99/22207

(51) Int. Cl. A61K 48/00; C12N 5/08; C12N 15/87

(52) U.S. Cl. 424/93.21; 435/455; 435/370

(57) ABSTRACT

Hepatocyte cells that are reversibly immortalized and grown in culture, which are functional and safe for use in transplantation, are disclosed. Also disclosed are methods for immortalizing primary hepatocytes, expanding the population of immortalized hepatocytes in culture, then reversing the immortalization to produce hepatocytes that are functional and safe for use in transplantation.

This application claims priority to U.S. Provisional Application No. 60/101,812, filed Sep. 25, 1998, and to copending PCT Application No. PCT/US99/22207 filed Sep. 24, 1999, each of which is incorporated by reference herein in its entirety.

Pursuant to 35 U.S.C. §202(c), it is acknowledged that the U.S. Government has certain rights in the invention described herein, which was made in part with funds from the National Institutes of Health, Grant Nos. R01-AI 31641 and DK48794-01.

### FIELD OF THE INVENTION

This invention relates to the treatment of liver disease and hepatic failure. In particular, the invention provides hepatocyte cells reversibly immortalized and grown in culture, which are functional and safe for use in transplantation.

### BACKGROUND OF THE INVENTION

Several scientific publications and patent documents are referenced in this patent application to describe the state of the art to which the invention pertains. Each of these publications is incorporated by reference herein, in its entirety.

Whole-organ liver transplantation is the current method of choice for treating patients with hepatic failure. Recent experimental evidence indicates that hepatocyte transplantation instead of whole organ transplantation can be used to treat hepatic failure and liver-based metabolic diseases, thereby obviating in some cases the need for surgery, with its associated risk. Hepatocyte transplantation also could be useful as a temporary treatment for patients with chronic liver failure, who are awaiting whole-organ transplantation. The first unequivocally successful heptaocyte transplantation into a human patient was recently reported (Fox et al., New Eng. J. Med. 338: 1422-1426, 1998), thereby demonstrating the clinical feasibility of this treatment method.

The shortage of human livers available as a source of hepatocytes for transplantation severely limits the use of this method for the treatment of liver failure. Animal hepatocytes could serve as an alternative, but significant concerns would exist with respect to transmission of infectious agents and immunological or physiological incompatibility with human hosts.

Another alternative to the transplantation of primary hepatocytes is the use of a clonal hepatocyte cell line that could be grown in culture and would exhibit the characteristics of differentiated, non-transformed hepatocytes following transplantation. Cloned hepatocyte cell lines have been developed by immortalization with a temperature-sensitive SV40 large T antigen (SV40Tag) (Fox et al., Hepatology 21: 837-845, 1995). These cells proliferate at the permissive temperature of 33° C. and lack characteristic features of differentiated hepatocytes. At the non-permissive temperature (37-39° C.), cell proliferation cease and the cells regain morphological characteristics of differentiated hepatocytes.

The aforementioned conditionally immortalized hepatocytes were found to function as well as primary hepatocytes following transplantation in rodents to reverse hyperammonemia-induced hepatic encephalopathy (Schumacher et al., Hepatology 24: 337-343, 1996) and to improve survival in experimentally-induced acute liver failure (Nakamura et al., Transplantation 63: 1541-1547, 1997). However, the continued presence of the oncogene (encoding SV40Tag) in these cells is of concern, inasmuch as it may increase the risk of malignant transformation following transplantation. A means to minimize or eliminate this risk heretofore has been unavailable.

### SUMMARY OF THE INVENTION

According to one aspect of the invention, a method of making a population of functional hepatocytes for transplantation into a patient is provided. The method comprises: (a) providing a sample of primary hepatocytes; (b) immortalizing the hepatocytes by transforming the hepatocytes with a vector comprising a removable DNA segment containing an oncogene, thereby producing immortalized hepatocytes; (c) growing the immortalized hepatocytes; and (d) removing the oncogene from the immortalized hepatocytes, the removal resulting in the production of the population of functional hepatocytes for transplantation into the patient. Preferably, the hepatocytes are obtained from a human donor and the oncogene is a gene encoding SV40 large T. antigen.

The oncogene is made removable by flanking it with recombinase target sites, and the removing is accomplished by introducing into the immortalized cells a gene that is expressed to produce a recombinase that specifically recognizes the recombinase target sites. Preferably, the recombinase is Cre recombinase and the recombinase target sites are loxP sites.

In preferred embodiments, the removable DNA segment further contains a suicide gene, which encodes a gene product that enables destruction of the immortalized cells by an exogenous agent if the removable DNA segment is not removed from the cells. the suicide gene preferably is a gene encoding herpes simplex virus thymidine kinase, and the cells are destroyed by exposure to gancyclovir if the removable DNA segment is not removed from the cells.

Another aspect of the invention provides a method of making a population of functional hepatocytes for transplantation into a patient, which comprises: (a) providing a sample of primary hepatocytes; (b) immortalizing the hepatocytes by transforming the hepatocytes with a vector comprising a removable DNA construct containing an oncogene, a selectable marker gene, and a gene encoding herpes simplex virus thymidine kinase, the genes together being flanked on either side by loxP sites; (c) growing the immortalized hepatocytes; and (d) reversing the immortalization of the hepatocytes by removing the DNA construct from the immortalized hepatocytes, the removing being accomplished by introducing into the immortalized hepatocytes a gene encoding Cre recombinase to effect excision of the DNA construct at the loxP sites, the excision resulting in the production of the population of functional hepatocytes for transplantation into the patient.

Populations of functional hepatocytes produced by the aforementioned methods are also provided, along with a method of treating a patient for hepatic failure, comprising transplanting into the patient a sufficient quantity of those hepatocytes to provide hepatic function to the patient.

According to another aspect of the invention, an immortalized hepatocyte is provided, which comprises a primary hepatocyte transformed with a DNA construct comprising two recombinase target sites that flank an oncogene which confers immortalization to the hepatocyte, wherein the immortalization is reversible by excision of the DNA construct by cleavage at the recombinase target sites when the target sites are exposed to a recombinase that specifically recognizes the target sites. Preferably, the recombinase target sites are loxP sites and the immortalization is reversible by Cre recombinase cleavage at the loxP sites. The DNA construct further includes a selectable marker gene, and may further comprise a suicide gene, which encodes a gene product that enables destruction of the immortalized hepatocyte by an exogenous agent if the DNA construct is not removed from the cells. Preferably, the suicide gene is a gene encoding herpes simplex virus thymidine kinase, and the exogenous agent is gancyclovir.

In one embodiment, the primary hepatocyte is obtained from a human donor, and the reversibly immortalized cell line, NKNT3, is provided. In another embodiment the primary hepatocyte is obtained from a rat donor, and the immortalized cell line, C8-B, is provided.

According to another aspect of the invention, a reverse-immortalized hepatocyte that is functional upon transplantation into a patient is provided, which is produced by exposing the DNA construct within the above-described immortalized hepatocyte to a recombinase that excises the DNA construct by cleavage at the recombinase target sites. A method of treating a patient for hepatic failure is also provided, comprising transplanting into the patient a sufficient quantity of the reverse-immortalized hepatocytes of claim 24 to provide hepatic function to the patient.

In another embodiment of the present invention, the cells of the invention are used to populate an ex vivo means for providing hepatic function. The cells may be attached, entrapped, immobilized or contained on or within a matrix or device. The methods of the invention are also used in other embodiments to generate cells or reverse-immortalized cells for use in populating or loading into matrices or devices disposed to receive cells, wherein the devices are used for providing in vivo or ex vivo support to hepatic function.

Other features and advantages of the present invention will be understood by reference to the drawings, detailed description and examples that follow.

### BRIEF DESCRIPTION OF THE DRAWINGS

FIG. 1. Schematic drawings of the integrating component of retroviral vector SSR69 before (upper) and after (lower) site-specific recombination. SSR69 contains the hygromycin B resistance gene (Hyg R) as a positive selectable marker and the herpes simplex virus thymidine kinase gene (HSV-TK) as a negative selectable marker. The Hyg R, HSV-TK and SV40 large T (SV40T) genes are flanked by loxP sites. The approximate locations of primers specific for SSR69 viral DNA (SSR-5′ and SSR-3′) and the neomycin resistance gene (NeoR, Neo-5′ and Neo-3′) are indicated. MoMLV, Moloney murine leukemia virus; LTR, long terminal repeat; IRES, internal ribosome entry.

FIG. 2. PCR and SQ-RT-PCR analysis of SSR69-immortalized hepatocytes (C8-B cells) before and after Ad-Cre infection. The PCR reaction used 4-fold serial dilutions of genomic DNA, isolated before (day 0) and 2 days after Ad-Cre infection (day2). The SSR69 (SSR-5′ and SSR3′) and NeoR-specific primers (which were used as a DNA loading control) are indicated in FIG. 1. SQ-RT-PCR reactions were performed using 4-fold serial dilutions of cDNA prepared with total RNA isolated before and 2, 5, and 7 days after Ad-Cre infection. Actin was used as a cDNA loading control and H₂O was used as a PCR control. Triangles represent the degree of serial dilution of DNA or cDNA used in each PCR or SQ-RT-PCR reaction.

FIG. 3. Growth kinetics and ³H-thymidine incorporation by C8-B cells. (FIG. 3A) The growth kinetics of C8-B cells were determined by plating cells at a density of 3×10⁴ cells per well in 6-well plates. Cells were then infected (solid square) with Ad-Cre or mock infected (day 0). Each time point represents an average from triplicate samples. (FIG. 3B) ³H-Thymidine incorporation was measured by incubating C8-B cells with 5 μCi/ml of ³H-thymidine at 37° C. for 2 hours before (day 0) and 1, 2, or 3 days after Ad-Cre infection. Results are shown as mean ±SD from three independent experiments, each performed in triplicate.

FIG. 4. Expression of liver-specific genes by C8B cells. Expression of the liver-specific genes, albumin (ALB), hepatocyte nuclear factor 4 (HNF4), UDP-glucuronosyltransferase-1 (UGT1), UDP-glucuronosyltransferase-2 (UGT2) and asialoglycoprotein receptor (ASGR), was analyzed by SQ-RT-PCR using total RNA isolated from C8-B cells before (day 0) and2, 5, and 7 days after Ad-Cre infection. Four-fold serial cDNA dilutions were used in the PCR reactions. H₂O was used as a negative PCR control, RNA isolated from primary rat hepatocytes was used as a positive control, and actin was used as cDNA loading control.

FIG. 5. Soft agar assay of C8-B/Ras cells. Anchorage independent growth of C8-B/Ras (SV40Tag⁺/ras⁺) cells was evaluated by soft agar assay. C8-B/Ras cells, mock infected or infected with Ad-Cre (indicated on the left) were incubated in the presence or absence of 5 μM gancyclovir (GCV, indicated on the top). Inserts are phase (40X) contrast micrographs of representative colonies.

FIG. 6. Principles of the procedure of reversible immortalization. The retroviral vector SSR69 (top) comprises the following elements from 5′ to 3′: (i) Moloney murine leukemia virus (MOMLV) long terminal repeat (LTR) with packaging signal (Ψ+), (ii) an initiation codon followed by a LoxP recombination target, whose overlapping open reading frame was fused to a hygromycin resistance/herpes simplex virus thymidine kinase (Hyg R/HSV-TK) fusion gene, (iii) the encephalomyocarditis virus internal ribosomal entry site (EMCV-IRES), which allows internal initiation of translation, (iv) the supertransforming U19 mutant of SV40T from which the intron was deleted to avoid splicing of the viral transcript and prevent expression of SV40 small t, (v) a second LoxP in direct orientation followed in frame by the neomycin resistance (NeoR) gene, but, importantly, lacking an initiation codon, and (vi) another LTR preceded by its polypurine track. Only HygroR/HSV-TK and SV40T are expressed in transduced cells in the absence of Cre recombinase. After Cre/LoxP recombination, the intervening DNA segment between the two recombination targets is excised (bottom right), so that only cells having excised SV40T become simultaneously G418 and ganciclovir resistant. (bottom left) Schematic representation of AxCANCre, the adenoviral vector expressing Cre recombinase.

FIG. 7. (FIG. 7A) Expression of proviral RNA encoding SV40T in NKNT-3 cells before and after Cre recombination and without or with G418 selection. (Top left) RT-PCR analysis with primers specific for SV40T without G418 selection of cells. (Top right) RT-PCR analysis with primers specific for SV40T after 7-day G418 selection of cells. (Bottom) β-actin loading controls for RT-PCR. Lane 1, no AxCANCre; lane 2, AxCANCre at MOI 5; lane 3, AxCANCre at MOI 10; and lane 4, AxCANCre at MOI 25. SV40T mRNA was markedly decreased after infection with AxCANCre at MOI 25 but could still be detected. However, SV40T mRNA could no longer be detected after AxCANCre infection and G418 selection. bp, base pairs. (FIG. 7B) (Top) Western blot analysis with SV40T monoclonal antibody after 7-day G418 selection of cells. (Bottom) β-actin loading controls for Western blot analysis; lanes 1, 2, 3, and 4 are as in FIG. 7A. SV40T protein could no longer be detected after AxCANCre infection and G418 selection. (FIG. 7C) Northern blot analysis of liver-specific mRNAs before (−) and after (+) Cre-mediated recombination of NKNT-3 cells; GAPDH was used as ‘housekeeping’ mRNA control. Data in FIGS. 7A, 7B and 7C were obtained from three independent experiments.

FIG. 8. Postoperative levels of total bilirubin (FIG. 8A), prothrombin time (FIG. 8B), and ammonia (FIG. 8C) and survival rate (FIG. 8D) in 90% hepatectomized rats. Group 1 (G1), no cell transplantation; group 2 (G2), intrasplenic transplantation of nonreverted NKNT-3 cells; and group 3 (G3), intrasplenic transplantation of reverted NKNT-3 cells (10 rats per group). Error bars indicate standard deviations. Statistical differences were determined by the Mann-Whitney U test, followed by the two-tailed Student's t test. The statistical analysis of survival time was done by the Kaplan-Meier survival test.

### DETAILED DESCRIPTION OF THE INVENTION

I. Definitions

Certain aspects of the present invention employ conventional molecular biology, microbiology, and recombinant DNA techniques that are well known in the art. See, e.g., Sambrook et al., “Molecular Cloning: A Laboratory Manual (1989); “DNA Cloning: A Practical Approach,” Volumes I and II (D. N. Glover ed. 1985); “Oligonucleotide Synthesis”(M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984); or “Current Protocols in Molecular Biology”, eds. Frederick M. Ausubel et al., John Wiley & Sons, 1999.

Therefore, if appearing herein, the following terms have the definitions set out below.

A “coding sequence” or “coding region” refers to a nucleic acid molecule having sequence information necessary to produce a gene product, when the sequence is expressed.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement other transcription control elements (e.g. enhancers) in an expression vector.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “nucleic acid construct” or “DNA construct” is sometimes used to refer to a coding sequence or sequences operably linked to appropriate regulatory sequences and inserted into a vector for transforming a cell. This term may be used interchangeably with the term “transforming DNA”. Such a nucleic acid construct may contain a coding sequence for a gene product of interest, along with a selectable marker gene and/or a reporter gene.

The term “selectable marker gene” refers to a gene encoding a product that, when expressed, confers a selectable phenotype such as antibiotic resistance on a transformed cell.

The term “reporter gene” refers to a gene that encodes a product which is easily detectable by standard methods, either directly or indirectly.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, the heterologous DNA is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A “vector” is a replicon, such as plasmid, phage, cosmid, or virus to which another nucleic acid segment may be operably inserted so as to bring about the replication or expression of the segment. More specifically, the term “viral vector” refers to a virus that is able to transmit foreign or heterologous genetic information to a host. This foreign genetic information may be translated into a protein product, but this is not a necessary requirement for the foreign information.

An “origin of replication” refers to those DNA sequences that participate in the in the initiation of DNA synthesis.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

The terms set forth below, relating to the biological molecules and methods of the present invention, are used throughout the specifications and claims.

The terms “immortalization” or “immortalized” refers to a cell, or a process for creating a cell, that will proliferate indefinitely in culture. In the present invention, immortalization refers to a process by which a primary cell culture is transformed in a way that causes the cells to behave in some respects like a tumor cell; specifically, in the proliferative characteristics of tumor cells.

The term “reverse-immortalization” refers to a process by which cells are immortalized by a means enabling them to be returned to a non-immortalized state at a later time.

A “reversibly immortalized” cell is a cell that is presently in an immortalized state, but can be returned to a non-immortalized state at a later time, utilizing the reverse-immortalization process described herein.

A “reverse-immortalized” cell is a cell that has been subjected to the entire process of reverse-immortalization, and now exists in a non-immortalized state.

The term “suicide gene” refers to a gene that confers a lethality phenotype to cells which are reversibly immortalized. In more common terms, the “suicide gene” can be thought of as a negative selectable marker gene. Expression of its gene product enables the cell to be killed, i.e., by treatment of the cell with an exogenous agent such as an antibiotic or antiviral agent.

The term “recombinase/recombinase target” refers to pairs of interacting molecules, one being a recombinase enzyme and the other being a DNA site specifically recognized and cleaved by that recombinase enzyme. The recombinase/recombinase target are paired by virtue of the specific interaction between the two, i.e., binding of the recombinase to its cognate DNA binding sequence, and cleavage of the DNA at that site.

The term “hepatic function” as used herein refers to a set of properties, qualities or behaviors of cells which are commonly understood by those skilled in the art to be functional, biochemical or genetic properties of hepatic cells or livers. Cells can be said to provide hepatic function or to be “functional” when they meet one or more of the following criteria: morphological and epithelial cell polarity characteristic of differentiated hepatocytes; production of liver-specific mRNAs; synthesis of proteins and enzymes typical of liver cells; ability to assist regulation of carbohydrate and fat metabolism; ability to assist detoxification of the blood; production of specific mRNA for albumin, or the corresponding protein; production of specific mRNA for hepatocyte nuclear factor, or the corresponding protein; production of specific mRNA for UDP-glucosyltranferase-1, or the corresponding protein; production of specific mRNA for UDP-glucosyltransferase-2, or the corresponding protein; production of specific mRNA for asialoglycoprotein receptor, or the corresponding protein; production of specific mRNA for androsterone-UGT, or the corresponding protein; production of specific mRNA for hepatic bilirubin-uridine-glucosyltransferase, or the corresponding protein; production of specific mRNA for glutathione S-transferase p, or the corresponding protein; production of specific mRNA for human blood coagulation factor X, or the corresponding protein; the ability to reduce bilirubinemia; the ability to reduce jaundice; the ability to improve hepatic encephalopathy; the ability to reduce hyperammonemia; the ability to produce clotting factors; or the ability to extend, beyond medical expectations the life of a patient with hepatic stress or failure due to acute liver failure, cirrhosis or other liver malfunctions or abnormalities.

II. Description

In accordance with the present invention, a means is now available for minimizing or eliminating the risk of malignant transformation of transplanted hepatocytes which have been produced by immortalization of primary hepatocytes and expansion in cell culture. The inventors have reversibly immortalized hepatocytes using a recombinant retrovirus containing an oncogene capable of inducing tumorigenic growth, flanked by recombinase target sites. Excision of the oncogene from the immortalized cells is accomplished by site-specific recombination following introduction into the cells of a gene encoding the recombinase that specifically recognizes the recombinase target sites. After site-specific recombination and oncogene excision, cell proliferation stops and the cells develop the characteristics of differentiated hepatocytes. Moreover, the cells possess minimal oncogenic potential as determined by in vitro assays. These cells have been transplanted into hyperammonemia-induced encephalopathic rodents and also into animals with experimentally-induced liver cirrhosis; the cells of the invention were capable of reversing these conditions.

The reverse-immortalized hepatocytes described above are superior to those heretofore produced because the risk of their malignant transformation in transplant patients is greatly reduced. The safe use of these cells for hepatocyte transplantation has been even further augmented in accordance with the invention, by the addition of a “suicide” gene to the initial retroviral construct used to immortalize the cells. In an exemplary embodiment, the suicide gene is a herpes simplex virus thymidine kinase (HSV-tk) gene, incorporated into the retroviral vector between the two loxP sites. If the oncogene is successfully excised by the Cre recombinase via the mechanism described above, the HSV-tk gene also is excised. If it is not excised, the cell can be destroyed by treatment with gancyclovir, an antiviral agent that targets the HSV-tk gene product.

Thus, the reversibly immortalized hepatocytes of the invention are hepatocytes that comprise a heterologous DNA construct comprising a selectable marker gene and an oncogene that enables the cells to proliferate in culture, the selectable marker gene and the oncogene together being flanked by DNA binding sites for a recombinase. Optionally, the DNA segment further comprises a “suicide” gene, also disposed within the recombinase binding sites. The invention is practiced by transforming the primary hepatocytes with the DNA construct, culturing the transformed hepatocytes under standard conditions suitable to expand the population of transformed hepatocytes, then exposing the transformed hepatocytes to the recombinase that recognizes the binding sites on the DNA construct (e.g. by infecting the transformed hepatocytes with a viral vector containing a gene encoding the recombinase). If the DNA construct also contains a “suicide” gene, the hepatocytes are further subjected to the conditions that will kill any cells still containing the DNA construct, following treatment with the recombinase.

Primary hepatocytes may be obtained from any donor or source. Preferably, the donor or source is a mammal, such as a mouse, and most preferably, the donor or source is a human. Methods for obtaining and initiating cultures of primary hepatocytes are well known in the art.

Any oncogene may be used to reversibly immortalize primary hepatocytes, and many of these are known in the art. The SV40Tag gene (a known oncogene used to immortalize primary cells) is preferred for use, but others may be used. These include, but are not limited to, viral oncogenes as known in the art, and cellular oncogenes such as mutant p53 genes or c-met genes encoding hepatocyte growth factor receptor among others.

The oncogene can be delivered by a variety of methods, for example, within any vector suitable for delivering genetic material to cells. Retrovirus vectors, specifically oncoretroviruses, are exemplified herein. Other suitable vectors include lentivirus vectors such as human immunodeficiency virus Type 1 (HIV-1), from which many suitable vectors have been developed. Other lentiviruses which are suitable for use as vectors include the primate lentivirus group including human immunodeficiency virus Type 2 (HIV-2)and human immunodeficiency virus Type 3 (HIV-3), simian immunodeficiency virus (SIV), simian AIDS retrovirus (SRV-1), and human T-cell lymphotropic virus Type 4 (HTLV4), as well as the bovine lentivirus, equine lentivirus, feline lentivirus, and ovine/caprine lentivirus groups. In addition to delivering the oncogene by the methods indicated above, the oncogene may be delivered by other means known in the art, for example, by electroporating into the cells in a manner similar to that taught in European Patent No. EP/0235113 (Henri et al, published Feb. 7, 1987).

Similarly, any selectable marker gene may be used in the DNA construct carrying the oncogene, and many of these are known in the art. Several selectable marker systems as well as vectors and other means for getting oncogenes into cells are described in “Current Protocols in Molecular Biology”, eds. Frederick M. Ausubel et al., John Wiley & Sons, 2001.

In addition many examples of suitable “suicide”genes are known in the art. The HSV-tk gene is preferred for use, but others may be used. Many examples are set forth by Ausubel et al., 2001, supra.

In addition to the immortalizing oncogene, selectable marker gene and the suicide gene, the DNA construct may comprise one or more desired genes, such as to promote growth or to provide a function reduced or missing from the donor's hepatocytes.

The aforementioned genes may be operably linked to one or more 5′ and/or 3′ expression-controlling regions, as are known in the art. With reference to promoters, constitutive or inducible promoters may be utilized, also as is known in the art.

DNA recombinase systems suitable for use in the invention are also known in the art. The cre/lox system (Cre recombinase, LoxP binding sites) is preferred for use, but other systems can also be used, including, but are not limited to the FLP/FRT system from Saccharomyces cerevisiae. It will be understood that if the DNA construct contains target binding sites for a particular recombinase, it is that recombinase that is to be used in reversing the immortalization of the hepatocytes.

U.S. Pat. No. 5,629,159 to Anderson describes a variety of DNA constructs exemplified for use in immortalization and dis-immortalization of pancreatic islet cells and neural cells. One or more of these variations may be adapted, in whole or in part, for reversible immortalization of hepatocytes in accordance with the present invention, using the methods described herein.

A preferred embodiment of the invention comprises (1) immortalizing primary hepatocytes with a retroviral vector containing the SV4oTag gene, the HSV-tk gene and a suitable selectable marker gene (e.g., neo or HSA, encoding the heat-stable antigen), flanked by loxP sites; (2) selecting transformants and growing them in culture; (3) reversing the immortalization by infecting the cells with an adenovirus vector carrying an expressible Cre recombinase gene to excise the oncogene; and, optionally, (4) destroying cells in which the oncogene was not successfully excised by treating the cells with gancyclovir. Vectors and systems of this type have been developed for reversible immortalization of various primary cells (Westerman & Leboulch, Proc. Natl. Acad. Sci. USA 93: 8971-8976, 1996), but have not been used for reversible immortalization of primary hepatocytes. More importantly, prior to the present invention, it was unknown whether such a system could be used to produce hepatocytes that would function in vivo following transplantation and that would be of sufficiently low oncogenic potential to be safe for such use.

The preferred embodiments have been tested, as described in detail in the examples. Briefly, hepatocytes were immortalized using a recombinant retrovirus containing the gene encoding SV40Tag flanked by loxP recombination target sites. Excision of SV40Tag from immortalized cells could then be accomplished by site-specific recombination with Cre-recombinase. Cells immortalized with this recombinant virus expressed SV40Tag and doubled in number every 48 hrs. After excision of the gene encoding SV40Tag with Cre-recombinase, cells stopped growing, DNA synthesis fell by 90%, and production of liver-specific mRNAs was either increased or became newly detectable. In addition, the morphology and epithelial cell polarity of the cells became more characteristic of differentiated hepatocytes. To determine their malignant potential, immortalized hepatocytes were transfected to express a second oncogene, activated H-ras. SV40Tag⁺/H-ras⁺-immortalized cells were capable of anchorage-independent growth and developed into tumors when injected in SCID mice. After SV40Tag excision using the Cre-recombinase, anchorage-independent growth stopped and tumor formation in SCID mice was abolished. Since immortalized hepatocytes also contained the gene encoding herpes simplex virus thymidine kinase, treatment with gancyclovir also produced complete regression of established tumors in mice.

Taken together, these results demonstrate that the reverse-immortalized hepatocytes produced by the method described above function in vivo as hepatocytes following transplantation. Moreover, the cells are of sufficiently low oncogenic potential to be safe for transplantation into patients in need of such treatment. Although similar reverse immortalization protocols have been employed for other cell types (e.g., pancreatic islet cells, muscle cells, neuronal cells), the successful reversal of immortalization to yield in vivo functional hepatocytes was an unexpected result. The aforementioned cell types were required to perform only one or two functions to be considered “functional” following reverse immortalization. For instance, muscle cells provide structural support. The predominant function of pancreatic islet cells is to produce insulin. Neuronal cells are judged by their ability to provide a structural neural connection. In contrast, hepatocytes perform a plethora of functions, many or all of which must be present in order for the cell to function in vivo. Such functions include synthesis of key proteins and enzymes, regulation of carbohydrate and fat metabolism and detoxification of the blood, among others. The inventors have provided evidence of differentiated hepatic function by the immortalized cells as evidenced by a variety of liver-specific functional proteins. The proteins represent a range of characteristics found only in differentiated hepatocytes. Albumin is uniquely produced by liver parenchymal cells and is a secreted protein. Androsterone-UGT is a microsomal protein that is expressed in differentiated hepatocytes only after birth and is an enzyme that degrades endogenous steroids. ASGPR, a plasma membrane protein, is lost in dividing cells and is the only liver specific protein whose expression is strongly controlled at the translational level.

Thus, the present invention demonstrates that, by transducing primary hepatocytes with a recombinant virus incorporating an oncogene, a suicide gene and a recombinase/recombinase target system, a well-differentiated, reversibly-immortalized non-tumorigenic hepatocyte cell line is generated. The successful generation of such a cell line would not have been predictable in advance of the results described in accordance with the present invention.

Hepatocyte transplantation holds great promise as an alternative to organ transplantation for patients with liver-based metabolic diseases and hepatic failure. A significant limitation to the development of this therapy relates to the limited availability of hepatocytes for transplantation. Theoretically, factors that limit the availability of solid organs for transplantation should not affect the availability of hepatocytes for this purpose. Primary hepatocytes have a tremendous capacity to proliferate in vivo and a small number of cells can be used to sequentially repopulate several generations of experimental animals whose liver cells are defective and can be replaced with unaffected donor hepatocytes. In addition, isolated liver cells can be cryopreserved for use when needed. Unfortunately, techniques for expanding isolated human hepatocytes in tissue culture and for cryopreservation of human hepatocytes heretofore were not adequately developed to be useful for human hepatocyte transplantation. Alternatives to the use of primary human hepatocytes for transplantation include the use of hepatocytes derived from other species and human hepatocytes conditionally immortalized for selective expansion in tissue culture.

In another embodiment of the present invention, the cells of the invention are used to populate an ex vivo means for providing hepatic function. The methods of the present invention are used to create cell lines which are used to populate an ex vivo device, alternatively, a quantity of cells may be grown and reverse-immortalized for loading into an ex vivo system. As will be appreciated by one of skill in the art, the cells may be attached, entrapped, immobilized or contained on or within the system, including in a matrix or device for the purpose of providing a populated device. In various embodiments, the populated devices are useful for short-term, long-term, or permanent replacement or supplementation of hepatic function. Such populated device means may be located within or as part of a larger device or they may be small and portable. In another embodiment, a device is sufficiently small that it may either be used ex vivo or placed within the body in an in vivo application. Such ex vivo populated devices may be long- or short-lived in terms of hepatic functionality and may be completely disposable. The populated devices may also be amenable to periodic repopulating, recharging or reactivating with further cells, and may include one or more replaceable cassettes, modules or cartridges for facilitating the repopulation operation.

The following examples are provided to describe the invention in greater detail. They are intended to illustrate, not to limit, the invention.

### EXAMPLE 1

### Reversible Immortalization of Rat Primary Hepatocytes using the Cre/Lox System

In the protocols described in this example, rat primary hepatocytes were immortalized using a recombinant Moloney-based retrovirus containing the gene encoding SV40Tag flanked by loxp sites. Cells were characterized before and after treatment with a recombinant adenovirus capable of transferring the gene encoding the Cre-recombinase to determine whether this approach could produce an hepatocyte cell line that would be useful clinically for transplantation.

### Materials and Methods

Animals. Inbred male Lewis rats (150-250 g) were obtained from Harlan Sprague-Dawley (Indianapolis, Ind.) and maintained in the Animal Resource Facility of the University of Nebraska College of Medicine (Omaha, Nebr.). Animals were maintained on standard laboratory chow on a 12-hour light/dark cycle. Severe combined immunodeficiency (SCID) mice were purchased from the Jackson Laboratory (Bar Harbor, Me.) and maintained in a barrier facility at the University of Nebraska College of Pharmacy. All procedures performed were approved by the University of Nebraska Institutional Animal Care and Use Committee and thus within the guidelines for human care of laboratory animals.

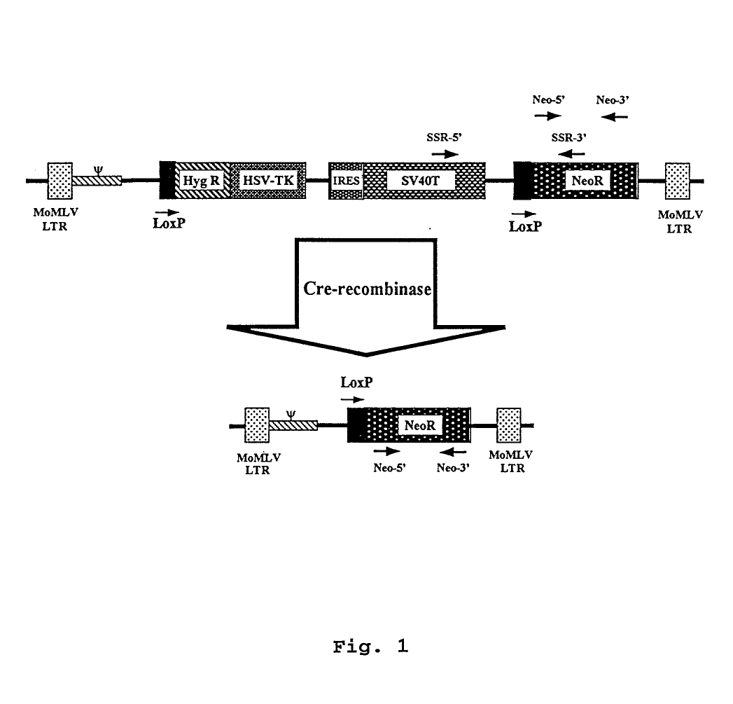

Recombinant Virus and Producer Cell Lines. The recombinant Moloney-based retrovirus SSR69 (FIG. 1), which contains the SV40Tag, herpes simplex virus thymidine kinase, and hygromycin resistance genes flanked by loxP sites has been described previously (Westerman KA, Leboulch P, Proc Natl Acad Sci U S A 1996; 93:8971-6). The SSR69 retrovirus producer cell line was maintained in Dulbecco's modified Eagle's medium (DMEM, Gibco/BRl, Gaithersburg, Md.) containing 10% newborn calf serum, 320 μg/ml Hygromycin B, and 1% penicillin/streptomycin (Gibco/BRl, Gaithersburg, Md.) and produces a virus titer of 1×10⁴ hygromycin-resistant cfu/ml when assayed on NIH 3T3 cells.

A recombinant adenovirus containing the gene encoding Cre-recombinase (Ad-Cre) was obtained. The transduction activity and titer of purified Ad-Cre virus stock was determined by counting G418-resistant cell colonies after infection of SSR69 producer cells with serial dilutions of virus stock.

Measurement of albumin production. To determine albumin production by immortalized hepatocytes, cells were plated into 12-well plates and incubated at 37° C. and 5% CO₂ in 0.3 ml/well of chemically-defined HGM media (Block GD, Locker J, Bowen WC, et al., J Cell Biol 1996; 132:1133-49). After 48 hours, the media was collected and assessed for albumin production by enzyme-linked immunosorbent assay (ELISA). ELISA was performed using a rabbit polyclonal anti-rat albumin capturing antibody (Cappel, Durham, N.C.) and a rabbit peroxidase-conjugated anti-rat albumin (Cappel, Durham, N.C.) secondary antibody. The standard curve was constructed using purified rat albumin (fraction V) purchased from Sigma Chemical Co. (St. Louis, Mo.).

Isolation and Immortalization of Lewis Rat Hepatocytes. Hepatocytes from Lewis rats were isolated by in situ collagenase (type I, Worthington Biochemical Corporation, freehold, N.J.) perfusion and plated on tissue culture flasks in Immortalization Medium [IM; DMEM containing 4% fetal calf serum, 0.2 μM Dexamethasone (Sigma Chemical Co., St. Louis, Mo.), and 1% penicillin/streptomycin supplemented with 10 μg/ml epidermal growth factor (EGF). After incubation for 24 hrs at 37° C. and 5% CO₂ , hepatocytes were transduced with the SSR69 retrovirus. Two days after infection, the EGF was removed and hygromycin was added to the culture media at 200 μg/ml. Hygromycin-resistant colonies emerged in 3 weeks. Individual colonies were isolated using cloning rings and expanded in culture at 37° C. and 5% CO₂. ³H-Thymidine uptake. Primary hepatocytes and immortalized hepatocyte clones were cultured on Primaria (Becton Dickinson Labware, Lincoln Park, New Jersey) plastic tissue culture plates at 37° C. and 5% CO₂ at a density of 3×10⁴ cells per well in 6-well plates. ³H-Thymidine incorporation was measured as previously described (Fox I J, Chowdhury N R, Gupta S, et al. Hepatology 1995; 21:837-46.). Briefly, cells were incubated with ³H-thymidine (5 μCi/ml) at 37° C. for 2 hrs. ³H-thymidine incorporation by primary hepatocytes was studied under the same conditions for comparison. The labeling medium was removed, the cells were washed with PBS and released with trypsin. Cells were then counted and trichloroacetic acid precipitable radioactivity was determined by scintillation counting using Hydrofluor (National Diagnostics, Manville, N.J.).

Determination of cell number: Primary hepatocytes and immortalized hepatocyte clones were plated at a density of 3×10⁴ cells per well in 6-well plates and cultured at 37° C. At 24 hr intervals, cells were released with trypsin and stained with trypan blue. Cell counts were determined using a hemocytometer

RNA isolation and semi-quantitative reverse transcription-polymerase chain reaction (SQ-RT-PCR). Total RNA was isolated by using Trizol reagent (Gibco/BRL, Gaithersburg, Md.) according to the manufacturer's recommendations. Expression of mRNA was analyzed by SQ-RT-PCR as described previously (Cai J, Phelan S A, Hill A L, Loeken M R. Diabetes 1998; 47:1803-5). Briefly, 200 ng of total RNA was reverse transcribed with random hexamer primers and serial dilutions of the resulting cDNA were amplified by PCR using sequence-specific primers.

SCID mouse studies. The tumorigenicity of cell lines was assayed by subcutaneous injection of cells into the flanks of immunodeficient (SCID) mice. Cells were dislodged from monolayer culture with trypsin, washed, and suspended in PBS. SCID mice were inoculated subcutaneously with 1×10⁶ cells per site. Animals were monitored every third day for the development of growth at the sites of injection. Animals were sacrificed at 24 weeks or when tumors reached 1 to 2 cm in diameter. Tumors were processed for routine histology and immunohistochemistry. To determine tumor sensitivity to treatment with gancyclovir, some animals were given a 0.2 ml intraperitoneal injection of gancyclovir (50 mg/kg) daily for 14 days when tumors reached 1-1.2 cm in size.

Immunohistochemistry was employed to determine SV40Tag expression in tumors. Unfixed frozen tumor sections were fixed in 1:1 acetone:methanol at −20° C. for 20 min. After incubation in blocking buffer (PBS +1% BSA), sections were incubated with mouse anti-SV40Tag mAb (CalBiochem, La Jolla, Calif.) followed by goat DTAF-conjugated anti-mouse IgG (Accurate Chemical and Scientific Co, Westbury, N.Y.).

Soft agar assay. To assay the capacity for anchorage independent growth, 5×10⁴ cells were suspended in 2 ml of 0.3 % Difco agar in IM and gently overlaid onto 60 mm dishes containing a lower layer of 0.5% agar. Cultures were fed every 5 to 7 days with a small amount of media.

After 20 days of growth at 37° C., the dishes were stained with 0.5 mg/ml p-iodonitrotetrazolium violet (Sigma Chemical Co., St. Louis, Mo.), and macroscopically visible colonies (>100 μm in diameter) were scored. HepGb2 cells were used as a positive control.

Production of activated H-ras immortalized hepatocytes. To establish an SV40Tag⁺/H-ras⁺-expressing immortalized hepatocyte cell line, a plasmid encoding activated H-ras and neo resistance, pSV2-Neo-EJ, was transfected into the SV40Tag immortalized hepatocytes by CaPO₄ precipitation. Two days later, transduced cells were selected in G418 (800 μg/ml). The resultant G418-resistant cells were then subcloned using cloning rings.

### Results

### Establishment of immortalized cell lines.

Following SSR69 retrovirus infection, transduced hepatocytes expressed SV40Tag, hygromycin resistance, and contained the gene encoding Herpes Simplex Virus thymidine kinase (HSV-tk). Hygromycin-resistant colonies emerged in 3 weeks and individual colonies were isolated using cloning rings.

Fifty cell clones were obtained and at least partially characterized. To determine which immortalized cell clones retained characteristics of differentiated hepatocytes, individual cell lines were screened by ELISA for secretion of albumin in the culture media. Twenty-four of the original 50 cell lines produced albumin when initially isolated.

The albumin-producing cell lines were then further characterized for additional evidence of hepatocyte-specific gene expression by RT-PCR using primers for the asialoglycoprotein receptor (ASGR), uridine diphosphate-glucuronosyltransferase-2 (UGT2), and hepatocyte nuclear factor 4 (HNF4). Eleven clones expressed only albumin mRNA, seven expressed ALB and one other liver-specific mRNA, three expressed 3 liver-specific mRNAs and three clones expressed all 4 mRNAs. Based on high-level albumin production and expression of all liver-specific mRNAs, one cell line (C8-B) was subcloned and used for further investigation.

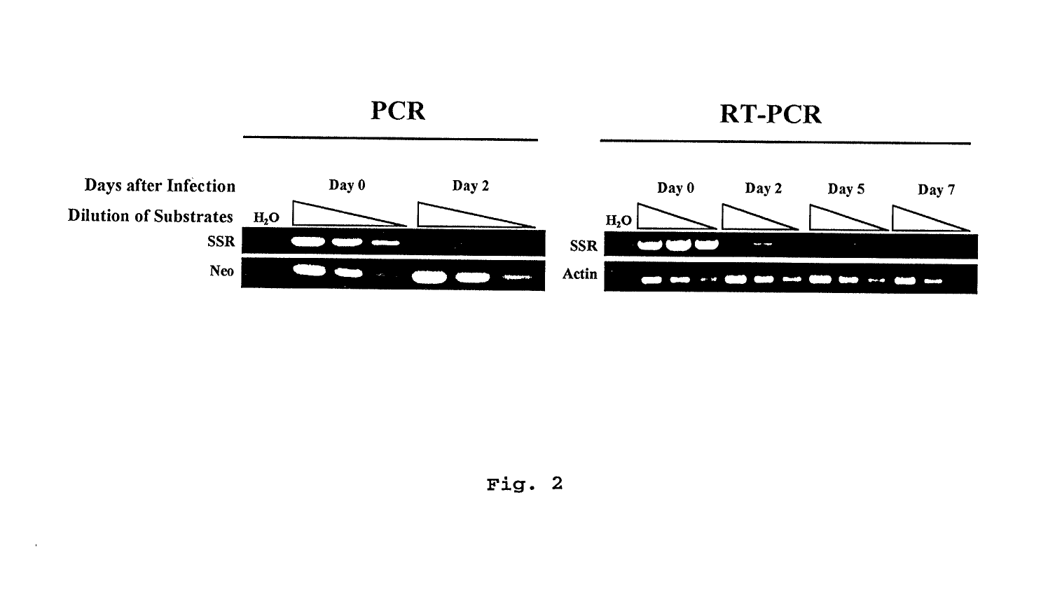

Excision of SV40Tag by site-specific deletion. To determine whether the gene encoding the SV40Tag could be efficiently deleted from immortalized C8-B cells, the cells were infected with Ad-Cre at an MOI of 1. Genomic DNA and mRNA were isolated at various time points after infection and analyzed by PCR and RT-PCR using SV40Tag/ SSR69 specific primers (FIG. 1). As shown in FIG. 2, the DNA fragment encoding the SV40Tag was completely excised by the Cre recombinase within 2 days of Ad-Cre infection. SV40Tag mRNA expression was ultimately eliminated; however, low level gene expression could still be detected up to 5 days after Ad-Cre infection. These results indicate that the gene encoding SV40Tag in SSR69-immortalized hepatocytes can be efficiently excised immediately after transduction with the Cre recombinase and that gene expression can be completely shut down using Cre/loxP-mediated site-specific recombination.

Cell Morphology. Light microscopy of the cultured immortalized cells was performed using phase contrast. Treatment with Cre recombinase changed the morphology of the C8-B cells. Six days after Ad-Cre infection, C8-B cells enlarged and increased their cytoplasm to nucleus ratio. Transmission electron microscopy of Ad-Cre treated and untreated cells revealed abundant mitochondria, lysosomes, rough endoplasmic reticulum and smooth endoplasmic reticulum. However, intercellular junctions with microvilli characteristic of bile canaliculi were seen in Ad-Cre treated cells only.

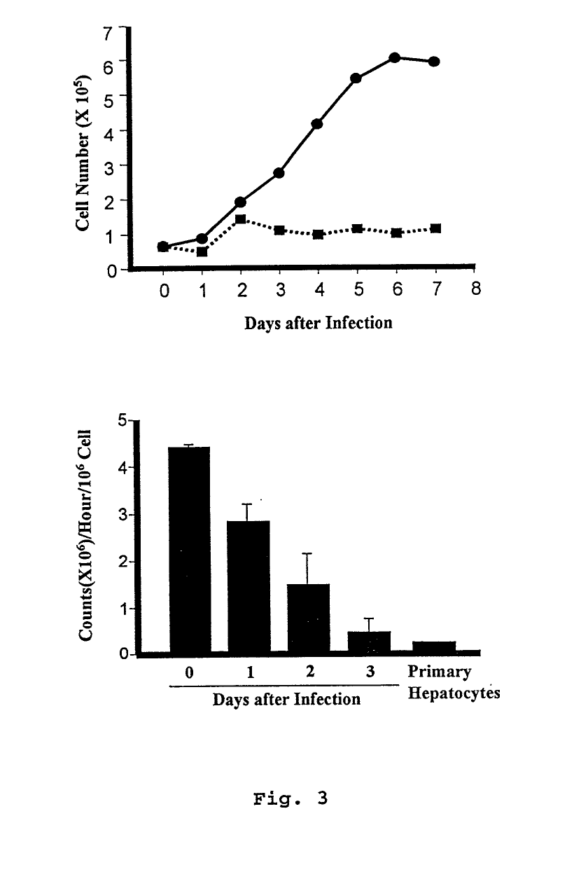

Cell proliferation and DNA synthesis. Before Ad-Cre treatment, immortalized cells doubled in number in approximately 48 hrs. After Ad-Cre infection, neither C8-B nor cultured primary hepatocyte cell numbers significantly increased (FIG. 3A).

FIG. 3B shows ³H-thymidine incorporation by cultured immortalized hepatocytes before and after Ad-Cre infection. The incorporation of ³H-thymidine by cells before Ad-Cre infection was 15-fold greater than that by freshly cultured primary hepatocytes (p<0.01 by Student's t-test). Three days after Ad-Cre infection, DNA ³H-thymidine incorporation was reduced to 1.5 fold that of primary hepatocytes which had been cultured for 24 hours.

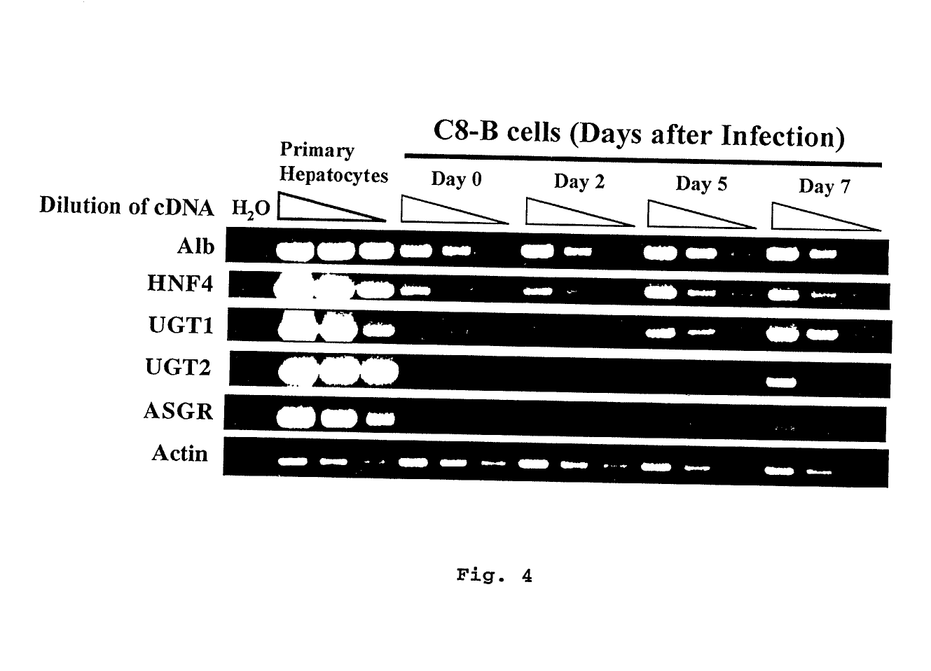

Liver-specific mRNA expression. To determine whether loss of the SV40Tag by site-specific recombination would produce a more differentiated phenotype, C8-B cells were assayed by SQ-RT-PCR before and after Ad-Cre infection for expression of liver specific mRNAs. Total cellular RNA was isolated before and 2, 5, and 7 days after Ad-Cre infection and reverse transcribed using random hexamer primers. The resulting cDNAs were then serially diluted and PCR amplified using sequence-specific primers for ALB (a secretory protein), ASGR (a cell surface protein), UGT1 (a microsomal protein responsible for glucuronidation of bilirubin), UGT2 (a microsomal protein responsible for glucuronidation of steroids), and HNF 4 (a transcription factor). RNA extracted from isolated primary rat hepatocytes was used as a positive control, and actin mRNA was used as an RNA loading control. As shown in FIG. 4, the mRNA level of all liver-specific proteins was increased. By day 5 to 7 after Ad-Cre infection, ALB, HNF 4 and UGT1 gene expression was detectable at a higher level than before Cre recombinase treatment, and UGT2 and ASGR mRNAs became newly detectable.

Malignant potential of SSR 69 immortalized hepatocytes. The malignant potential of C8-B cells was first determined by using a soft agar assay. The result showed that C8-B cells produced no anchorage independent colonies, but grew as small clusters that expanded in only two dimensions. This contrasts markedly with the growth pattern of HepGb2 cells that grew into large 3-dimensional colonies and continued to enlarge exponentially with time. The malignant potential of C8-B cells, which still produce SV40Tag, was further analyzed by transplanting them into the flanks of SCID mice. Subcutaneous implantation of 1×10⁶ cells produced no tumors after 24 weeks of implantation, whereas injection of the same number of HepGb2 cells produced large tumors within 4 weeks of implantation (data not shown).

Since transfection of SV40 immortalized hepatocytes with activated c-Ha-ras has been shown to transform them into strongly tumorigenic cells which are histologically poorly differentiated, C8-B cells were transduced to express the activated ras gene in order to assess whether mutational activation by a second transforming gene could produce malignant transformation of SSR 69-immortalized cells. Cells were transfected with plasmid pSV2-Neo-EJ and individual G418-resistant cell colonies were cloned and analyzed for expression of H-ras and SV40Tag genes by RT-PCR. One cell line, which expressed both H-ras and SV40Tag (C8-B/Ras), was used for further study.

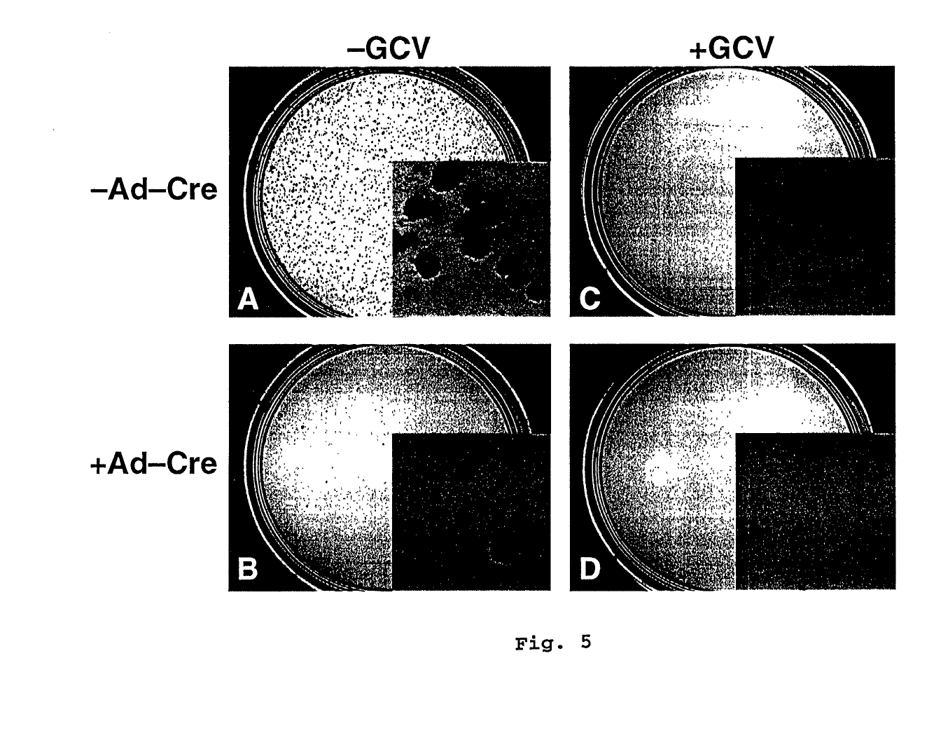

As shown in FIG. 5, C8-B/Ras cells developed anchorage-independent large colonies in soft agar. Since these cells contain the gene encoding the herpes simplex virus thymidine kinase, colonies were assessed for sensitivity to treatment with gancyclovir and could be eliminated in media containing 5 μM gancyclovir. Following infection with Ad-Cre, soft agar culture produced only rare three-dimensional colonies. These colonies resulted from failure to undergo site-specific recombination since all were sensitive to gancyclovir and, as determined by PCR, still contained the genes encoding the SV40Tag and H-ras (data not shown)

Tumor potential was further assessed by inoculating C8-B/Ras cells into SCID mice. C8-B/Ras cells induced tumors at all inoculated sites, and grew to more than 1 cm within 3 weeks. Following SV40Tag excision with Ad-Cre, tumors developed in only one of four inoculation sites and reached 1.5 cm in size only after 8 weeks. Immunohistochemistry showed that this tumor grew from cells which had not undergone recombination since all tumor cells stained positively for SV40Tag (data not shown).

When tumor-bearing animals were given a 14-day course of gancyclovir (50 mg/kg), tumors (>1 cm in size) stopped growing within 5 days of beginning therapy, and could no longer be identified clinically or histologically by the end of treatment. In addition, no tumor recurrence occurred during a 4-week post-gancyclovir observation period.

### Discussion

We have previously demonstrated that primary rat hepatocytes, immortalized with a thermolabile mutant SV40 large T antigen can maintain a significantly differentiated hepatic phenotype in culture and can function as well as primary hepatocytes following transplantation in animal models of liver-based metabolic disease and liver failure. Those cells did not demonstrate anchorage independent growth in tissue culture and, when transplanted into syngeneic rats or immunodeficient mice, did not form tumors. Down-regulation of the mutant thermolabile large T antigen at physiologic temperature provided a minimal degree of protection from the development of tumors in animals receiving the transplants. However, a variety of factors could potentially affect the function and growth of such cells.

To provide more stringent control over expression of the transforming gene, the loxP/Cre system was employed. In this study, the gene encoding the SV40Tag was completely eliminated from C8-B cells within two days of Ad-Cre infection. After the gene was deleted, the cells stopped growing, their DNA synthesis fell, their expression of liver-specific mRNAs increased, and they regained the morphological appearance of differentiated hepatocytes. Thus, reversal of immortalization was accomplished with complete removal of the offending transforming gene.

While C8-B cells were not tumorigenic by in vitro assay or following transplantation, when transduced to express a second transforming gene, SV40Tag⁺/H-ras⁺cells formed large colonies in soft agar and developed into tumors in SCID mice. After Ad-Cre infection, only occasional SV40Tag⁺/H-ras⁺C8-B cells formed anchorage-independent cell colonies in soft agar and produced slow growing tumors in SCID mice. These colonies and tumors, however, were sensitive to treatment with the antiviral agent gancyclovir. Since the gene encoding HSV-tk in the immortalized cells is flanked by loxP sites, SV40Tag-deleted cells are not sensitive to gancyclovir. Therefore, gancyclovir could be administered to transplant recipients to eliminate engrafted SV40Tag-expressing immortalized hepatocytes but would not eliminate engrafted SV40Tag-deleted cells in recipients treated with gancyclovir. The recombinant vector used to make the immortalized hepatocytes in these experiments produced cells which express the neomycin-resistance gene following recombination. This vector could easily be redesigned so that transduced cells express green fluorescent protein upon recombination. Thus, it would be possible to select cells for transplantation which have undergone recombination based on their fluorescence characteristics.

### EXAMPLE 2

### In vivo Characteristics and Function of loxP/Cre Generated Reversibly Immortalized Rat Hepatocytes

The functional capacity of 24 individual hepatocyte cell clones that secreted albumin into the culture media was then assessed in an experimental model of inducible hepatic encephalopathy. Total portacaval shunts (PCS) were performed on Lewis rats that were then subjected to ammonium acetate administration. PCS rats that receive exogenous ammonium acetate become hyperammonemic and comatose within30 minutes (Conjeevaram et al, Hepatology 19: 1245-1250, 1994; Ribiero et al., Hepatology 15:12-18, 1992; Blei et al., Hepatology 19: 1437-1443, 1994). Encephalopathy can also be quantified by means of a coma scale based on evaluation of a variety of behavioral reflexes (Maximum: 15 points indicates normal behavior) (Rigotti et al., Arch Surg. 120: 1290-1295).

Two weeks after PCS, 3×10⁷ cells from each clone, including C8-B cells, were transplanted by injection into the inferior pole of the spleens of one to two Lewis rats. Interestingly, only three of the 24 clones, not including C8-B, were capable of affecting the plasma ammonia level and hepatic encephalopathy score of portocaval shunted animals following ammonium acetate administration. Thus, the functional capacity of the cell clones did not directly correlate with the degree of liver-specific mRNA expression as determined by the previous RT-PCR analysis. One clone, RH69 was then selected for detailed analysis based on the results of the initial screening.

Four experimental groups were used for this analysis. These included 1) animals that underwent portacaval shunt only (PCS) (n=4); 2) animals that underwent portacaval shunt and intrasplenic transplantation of 3×10⁷ primary hepatocytes 2 weeks after shunt surgery (pHTx) (n=5); 3) animals that underwent portacaval shunt and intrasplenic transplantation of 3×10⁷ RH69 cells 2 weeks after shunt surgery (iHTx) (n=12); and 4) animals that underwent portocaval shunt and intrasplenic transplantation of 3×10⁷ Ad-Cre infected RH69 cells (no longer expressing the SV40Tag) 2 weeks after shunt surgery (riHTx).

Thirty minutes after injection of 3.4 mmol/kg ammonium acetate, plasma ammonia levels were elevated in all rats tested. All the animals that underwent cell transplantation, however, were protected from the development of significant hyperammonemia compared to control animals (PCS only: 1190±260; pHTx: 330±40; iHTx: 470±110; riHTx: 585±102 mmol/l. p=NS pHTx vs iHTx and riHTx; p<0.01 PCS vs all HTx groups).

The results of the behavioral tests following ammonium acetate administration paralleled the plasma ammonia levels. PCS rats that had not undergone hepatocyte transplantation lost their righting reflex within 8 minutes of ammonium acetate administration and developed progressive neurological deterioration. In contrast, animals that had undergone PCS and cell transplantation showed only a slight alteration in their encephalopathy scores when assessed thirty minutes after ammonium acetate exposure. At four weeks after transplantation, PCS only: 1.3±0.5; pHTx: 12.4 ±1.7; iHTx: 11.0±2.6; riHTx: 10.8±1.5; p<0.05 PCS vsall HTx groups; p=NS pHTx vs iHTx and riHTx).

Reversal of protection from encephalopathy following splenectomy. In contrast to when hepatocytes are transplanted into the spleens of normal rats, the total diversion of portal blood to the vena cava in PCS rats precludes the migration of the transplanted cells from the spleen to the liver. To assess whether the hepatocytes that were engrafted within the spleen were responsible for the biochemical and behavioral changes in PCS rats, the transplant recipient rats were subjected to splenectomy. One week after splenectomy, ammonium acetate administration led to a significant elevation in plasma ammonia level in all the transplanted animals (pHTx: 330±40 vs. 1370±680; iHTx: 470±70 vs. 930±90; riHTx: 590±100 vs. 1280±470 mmol/l; p<0.01 all groups) and a marked reduction in encephalopathy score compared to pre-splenectomy levels (pHTx: 12.4±1.7 vs. 1.2±0.8; iHTx: 11.0±2.5 vs. 1.8±0.5; riHTx: 11.5±2.5 vs. 1.3±0.5; p<0.01 all groups), indicating that the cells resident in the spleen were the only significant source of metabolic support in this system.

PAS and hematoxylin and eosin staining was performed on sections of spleens removed three months after hepatocyte transplantation. These studies showed that mature hepatocytes were implanted in the red pulp and had normal nucleus to cytoplasm ratios and that transplanted primary hepatocytes were indistinguishable from transplanted immortalized hepatocytes.

Immunohistochemical staining of frozen spleen sections revealed SV40Tag staining from only the spleens of animals transplanted with immortalized hepatocytes not infected with the Ad-Cre virus. No hepatocytes of abnormal morphology or tumors were found in rats during the 12-month observation period after transplantation (Cai et al., 2001, ASTS Meeting, Chicago, Ill., Transplantation, Abstracts).

Reversal of protection from hyperammonemia and encephalopathy following gancyclovir treatment. Since the immortalized hepatocytes contain the genes encoding the SV40Tag and HSV-tk flanked by loxP sites, cells that have not been infected with Ad-Cre express both genes and would be sensitive to treatment with gancyclovir (GCV). To examine whether transplanted hepatocytes that had not excised SV40Tag following Ad-Cre treatment could be eliminated by gancyclovir infusion, portocaval-shunted rats that had 1) undergone stable transplantation with RH69 cells or Ad-Cre treated RH69 cells and 2) subsequently been protected from hyperammonemia-induced encephalopathy were subjected to a daily intraperitoneal injection of gancyclovir at 50 mg/kg.

Following two weeks of gancyclovir treatment, animals underwent ammonium acetate challenge and were assessed for protection from hyperammonemia and hepatic encephalopathy. Gancyclovir produced a significant change in ammonia level (iHTx: 420±60 before gancyclovir administration vs 1490±80 after gancyclovir administration, p<0.01) and encephalopathy score (iHTx: 10.8±1.5 before gancyclovir administration vs 1.8±0.9 after gancyclovir administration, p<0.01) in animals transplanted with RH69 cells that had not been previously infected with the Ad-Cre virus (to excise the SV40Tag and HSV-tk genes) but produced no change in ammonia level (riHTx: 590±100 before gancyclovir administration vs 540±50 after gancyclovir administration, p=NS) or encephalopathy score (riHTx: 11.3±1.0 before gancyclovir administration vs 10.5±1.7 after gancyclovir administration, p=NS) in animals transplanted with RH69 cells that had undergone excision of the SV40Tag and HSV-tk genes. These studies indicate that the HSV-tk gene can be used to safeguard against the transplantation of hepatocytes still expressing the SV40Tag by eliminating only the “unreversed” conditionally immortalized cells (Cai et al, supra).

### EXAMPLE 3

### Reversibly Immortalized Rat Hepatocyte Transplantation Enhances Liver Function and Survival in Cirrhotic Rats

Hepatocyte transplantation was shown to be effective in treating liver failure in cirrhotic rats. A determination was made as to whether reversibly immortalized hepatocytes could improve liver function and survival in a model of liver failure physiologically identical to that seen clinically.

Development of a reproducible model of end-stage liver disease secondary to cirrhosis in rats. None of the classic rodent models for acute liver failure or encephalopathy directly correlate with the physiologic abnormalities associated with the chronic liver dysfunction that is seen in patients. In order to address the role of hepatocyte transplantation in treating decompensated chronic liver disease, liver cirrhosis was induced in rats using phenobarbital (Sigma Chem. Co. St. Louis, Mo.) and carbon tetrachloride (CCl₄, Sigma). Lewis rats were given phenobarbital (0.5 g/L) added to the drinking water. Two weeks later, CCl₄ (diluted 1:9 in olive oil) was given on a full stomach intragastrically by gavage twice a week. The initial dose was 0.2 ml/kg but each subsequent dose was adjusted weekly based on changes in body weight. When there was no change or an increase in body weight, CCl₄ was given at 0.2 ml/kg. When body weight decreased 1-5 g, CCl₄was given at 0.15 ml/kg. When body weight decreased by 6-10 g, CCl₄was given at 0.1 ml/kg and when body weight decreased >11 g, CCl₄ was not given and the CCl₄ dose was reassessed one week later. When rats developed ascites, laboratory tests were drawn and repeated weekly. When rats developed ( 1) plasma total bilirubin >0.5 mg/dl (normal <0.1 mg/dl), ( 2) prothrombin time (PT) >14.5 sec, ( 3) plasma ammonia >145 mmol/l and ( 4) persistent clinically proven ascites, phenobarbital and CCl₄ were discontinued. Only rats whose measures of liver function did not return toward normal four weeks after discontinuing of phenobarbital and CCl₄ were subjected to transplant experiments. Irreversible liver cirrhosis required 26-28 weeks of treatment (Kobayashi et al., Hepatology 31: 851-857, 2000).

In initial and ongoing studies, two cirrhotic rats were treated by intrasplenic transplantation of 50×10⁶ RH69 loxP/Cre generated reversibly immortalized rat hepatocytes. Both animals died within 5 days of intervention. Histologic examination of autopsy specimens demonstrated a few RH69 cells engrafted in the spleen but, more importantly, a large number of transplanted RH69 cells were found occluding the portal vein in the substance of the liver.

Since the transplanted RH69 cells used for these studies had not been treated with Ad-Cre, the cells were morphologically smaller than primary rat hepatocytes and were, therefore, more likely to have traversed the splenic sinusoids and entered the portal circulation.

In order to avoid translocation of transplanted RH69 cells into the portal circulation and enhance engraftment in the spleen, the RH69 cells were encapsulated using sodium alginate. Sodium alginate is derived from the brown seaweed, Laminaria hyberborea, and has been used extensively in cellular transplant studies where encapsulation is required. The RH69 cells were encapsulated with a 10% sodium alginate solution in a 1:4 (packed cell volume: alginate volume) ratio for transplantation. In initial studies, three cirrhotic rats were treated by intrasplenic transplantation of 50×10⁶ RH69 loxP/Cre-generated reversibly immortalized rat hepatocytes. Two weeks after hepatocyte transplantation, measures of liver function were significantly different. The PT had fallen from 26.2±14.3 to 14.9±2.8 seconds, the TB had fallen from 0.8±0.3 to 0.3±0.1 mg/dl, alb increased from 2.5±0.2 to 2.8±0.2 g/dl and hepatic encephalopathy (HE) score increased from 6.7±0.6 to 12.0±1.0 (no encephalopathy =15). Survival is also prolonged so far, in that all transplanted animals have already survived more than 36 days (compared to controls 15.5±4.9 d).

### EXAMPLE 4

### Reversibly Immortalized Human Hepatocytes Prevent Acute Liver Failure in Rats

### Materials and Methods

### Cell Lines and Creation of NNKT3 Line

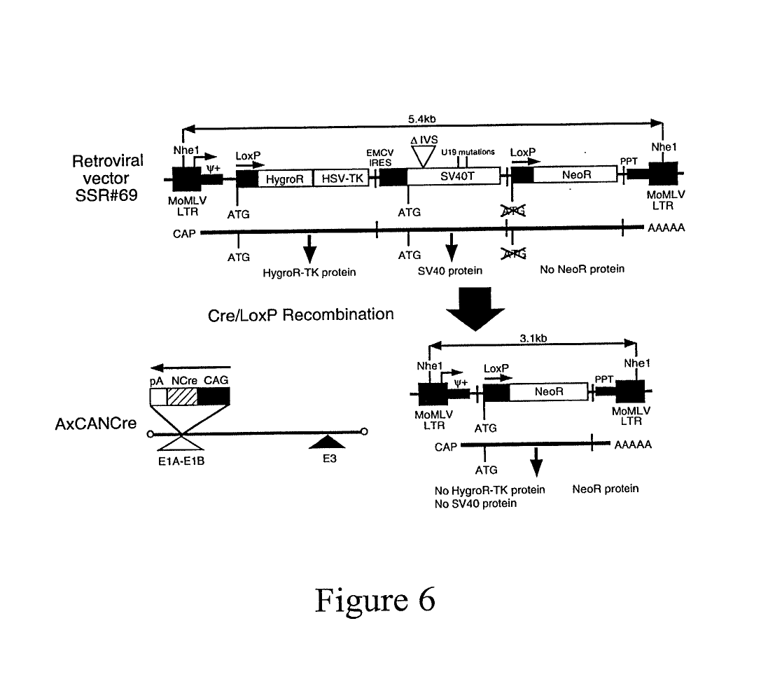

Retroviral Vector. A polycistronic retroviral vector SSR 69 (Westerman and Leboulch, PNAS 93: 8971, 1996) was constructed to transfer and express the simian virus 40T (SV40T) immortalizing gene flanked by LoxP recombination targets (FIG. 6).

Transduction of hepatocytes. Primary adult human hepatocytes were submitted to the reversible immortalization procedure upon transduction with SSR#69 virions produced in an amphotropic packaging cell line free of replication-competent retrovirus. The amphotropic TCrip packaging cell line producing SSR 69 virus was grown as described in (Westerman and Leboulch, 1996, supra). Adult human hepatocytes and the serum-free culture medium, CS-C, were purchased from Cells Systems (Seattle, Wash.). Cells were transduced with 2 ml of ψCrip cell supernatant per T75 flask in the presence of polybrene (12 μg/ml) at 37° C. twice for 4 h. Two days after transduction, selection was applied with CS-C medium containing hygromycin(320 μg/ml). Hygromycin-resistant hepatocyte clones were isolated with cloning rings 5 weeks after the start of selection. Viral titers were 5×10⁴ plaque-forming units (PFU)/ml as assessed on NIH3T3 cells after selection with hygromycin as described in (Westerman and Leboulch, 1996, supra). One of the resulting immortalized clones, referred to as the NKNT-3 cell line, was chosen for further analysis on the basis of growth characteristics and liver-specific functions.

### Surgical Procedures

90% Hepatectomy. Ninety percent hepatectomy was done by removing the median, left lateral, right upper and and lower lobes by ligation leaving only the caudate lobe. After surgery, animals were allowed free access to drinking water supplemented with 10% dextrose. Animals also received a daily intravenous injection of 2 ml of saline.

Intrasplenic Transplantation of Reversibly Immortalized Hepatocytes. A small surgical incision was made in the animal's flank, and the spleen was exposed under anesthesia and surgical care approved by the animal committee of Okayama University Medical School. Cells (50×10⁶) suspended in 0.5 ml of ASF-104 were injected into the inferior pole of the spleen. The blood flow in the splenic artery and vein was temporarily occluded to avoid immediate passage of cells into the portal vein during transplantation. The injection site was also ligated to prevent cell leakage and bleeding.

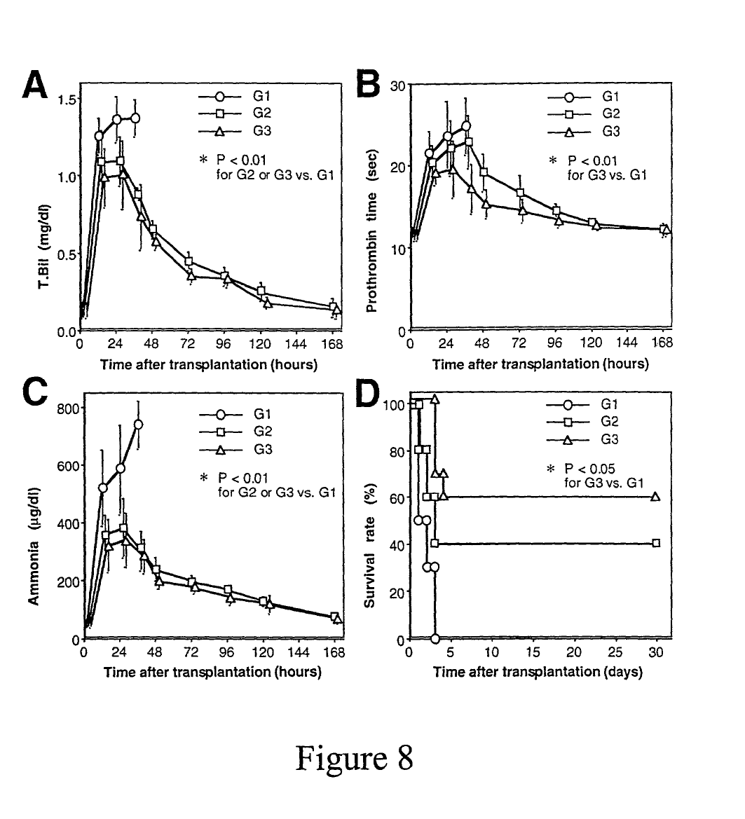

Biochemical Blood Analysis. Blood samples were obtained from tail veins, post-operatively. Biochemical parameters that include total bilirubin (T.Bil), prothrombin time (PT), and blood ammonia (NH3)were measured in the plasma of transplanted and hepatectomized rats at various time points (FIG. 8 A, B, and C). T.Bil, PT, and NH3 determinations were made with Fuji Dry Chem (Tokyo, Japan).

### Analysis of NKNT-3 Cell Line

Histology: Spleen specimens were compound embedded and frozen at −80° C. Cryostat sections of the spleen(5 mm thick) were fixed in ice-cold acetone. Immunofluorescence for SV40T of NKNT-3 cells transplanted into the spleen was performed as the same procedure as described in Kanegae et al.(Nucleic Acids Research 23: 3816, 1995).

Immunofluorescence. For the detection of SV40T by indirect immunofluorescent staining, NKNT-3 cells (5×10⁴)were grown overnight on sterile slides at 37° C., rinsed with phosphate-buffered saline (PBS) solution, and fixed in cold acetone. Samples were blocked with 10% fetal bovine serum in PBS for 1 h at room temperature. Primary antibody was mouse monoclonal immunoglobulin G2a (IgG2a) antibody to SV40T (Santa Cruz Biotechnology, Santa Cruz, Calif.),used at a concentration of 100 μg/ml, incubated with sample overnight at 4° C. IgG-fluorescein isothiocyanate rabbit polyclonal antibody to mouse IgG (Santa Cruz Biotechnology; Santa Cruz, Calif.) was subsequently used at a concentration of 10 mg/ml and incubated for 1 h at 37° C.

Tumorigenicity. NKNT-3 cells suspended in 0.5 ml of ASF-104 medium were subcutaneously injected into the dorsal midline of five 6- to 8-week-old SCID mice. Mice were observed for 2 months after injection.

MTT Cytotoxicity Assays. Sensitivity of NKNT-3 cells to either 5 μM ganciclovir or G418 (500 μg/ml) was confirmed by 3-(4,5-dimethyl-2-thiazolyl)-2,5-diphenyl 2Htetrazolium bromide (MTT) cytotoxicity assays. NKNT-3 cells were plated in a 96-well microplate at 5×10³ cells per well to obtain a growth curve. On days 1, 3, 5, and 7, MTT (20 μg/ml) was added to a different well, incubated for 4 h, and then reacted with 150 μl of isopropanol for 10 min. Relative percentage of viability was determined by the ratio of absorbance at 570 and 630 nm with Bio-Rad EIA reader (Bio-Rad, Richmond, Calif.).

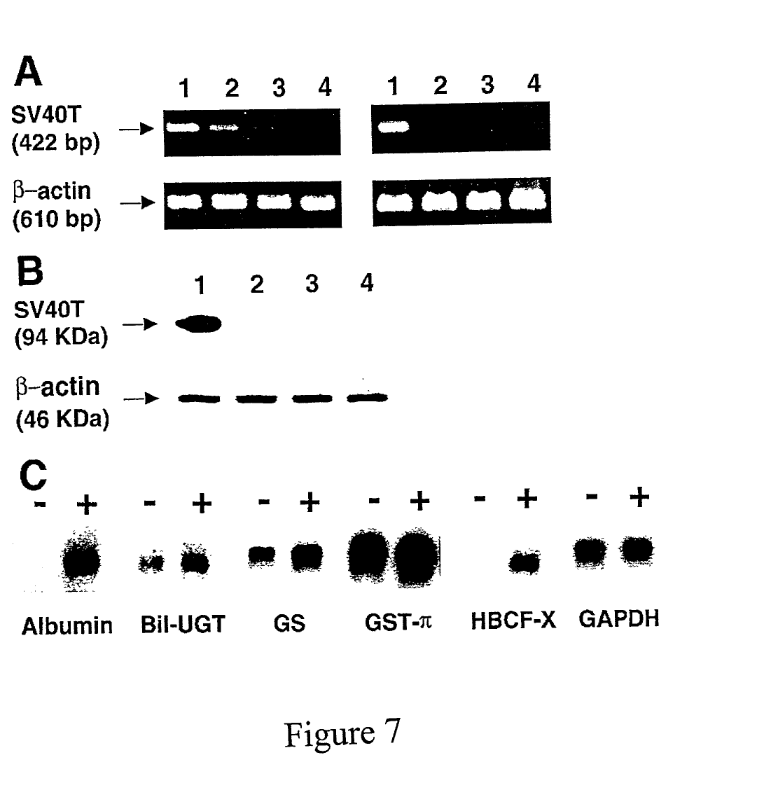

Northern Analysis of Specific Transcripts. Northern blot analysis was performed as described (Westerman and Leboulch supra). Specific DNA probes were obtained by PCR of genomic DNA and then radiolabeled. The expression of key genes of liver metabolism was assessed by Northern blot analysis: albumin, GS, hepatic bilirubin-uridine diphosphate- glucuronosyltransferase (Bil-UDT), glutathione S-transferase p (GST-p), and human blood coagulation factor X (HBCF-X); the house-keeping mRNA glyceraldehyde-3 phosphate dehydrogenase (GAPDH) was used as an internal control (FIG. 7C).

NKNT-3 cells (6×10⁶) were plated in T75 flasks and infected 1 day later with AxCANCre at various MOI for 1 h. Cells were subsequently cultured in the chemically defined serum-free medium ASF-104 (Ajinomoto, Tokyo, Japan) for 2 d and then harvested for RT-PCR, Western blot, and Northern blot analyses. In other experiments, infection of AxCANCre was performed as above. After adenoviral infection, NKNT-3 cells were cultured in ASF-104 medium containing G418 (500 μg/ml) for 7 d and then harvested for RT-PCR, Western blot, and Northern blot analyses.

Reverse Transcription(RT)-Polymerase Chain Reaction (PCR). Total RNA was isolated by the RNAzol procedure (Cinna/BioTecx, Friendswood, Tex.). RT was performed at 22° C. for 10 min and then 42° C. for 20 min with 1 μg of RNA per reaction. PCR was performed with specific primers in volumes of 50 μl and according to the manufacturer's instructions (PCR kit; Perkin-Elmer/Cetus, Norwalk, Conn.). Primers designed to identify the presence of SV40T and β-actin were constructed. PCR conditions were as follows: denaturation at 92° C. for 1 min, annealing at 58° C. for 1 min, and elongation at 72° C. for 1 min with a thermal cycler (Perkin-Elmer, Foster City, Calif.). PCR products were resolved on 2% agarose gels and visualized by ethidium bromide staining.

Western Analysis. Proteins (30 μg per lane) from NKNT-3 cells and reverted NKNT-3 cells were separated by electrophoresis on SDS-polyacrylamide gels, transferred to hybond-polyvinylidene difluoride transfer membranes, and treated with mouse monoclonal antibody to SV40T (Santa Cruz Biotechnology) (1:100) followed by peroxidase-linked secondary antibody(1:2500). Labeled protein bands were stained with ECL kit (Amersham, Japan). Human β-actin protein served as an internal control.

### Transplant Experiments

Lewis rats weighing 350 g were used in transplant experiments and divided into the following groups: group 1 (G1: n=10), intrasplenic injection of 0.5 ml of medium; group 2 (G2: n=10), intrasplenic transplantation (Isp-Tx) of 5×10⁷ nonreverted NKNT-3 cells; and group 3 (G3: n=10), Isp-Tx of 5×10⁷ NKNT-3 cells treated with AxCANCre at MOI 10 and subsequent G418 (500 μg/ml) selection for 7 d. All animals underwent 90% hepatectomy 1 d after transplantation and received a daily intramuscular administration of FK506 (1 mg/kg) to prevent rejection of human xenotransplanted cells. Transplantation of normal rat hepatocytes was not performed as a control in the present experiments, because the beneficial effect of transplanting primary hepatocytes into the spleen has been shown by many investigators in the rat model of acute liver failure (ALF) (Demetriou,1986; Arkadopoulos et al.,1998; Schumacher et al., 1996; Nakamura et al., 1997). If the animals did not die of ALF during the first 3 d after hepatectomy, the rats were monitored for four weeks after hepatectomy, at which time liver regeneration sufficient to ensure long-term survival was achieved.

### Results

In the retroviral vector, SSR#69, a protein fusion, referred to as Hygro-TK, is concurrently expressed and confers both resistance to hygromycin and sensitivity to ganciclovir (FIG. 6). After transient expression of the Cre recombinase, precise recombination occurs between LoxP sites within the chromosomally integrated provirus (FIG. 6). As a consequence, both Hygro-TK and SV40T genes are permanently excised from the genome, whereas the neomycin resistance gene (NeoR), which confers resistance to G418, becomes activated (FIG. 6).

A reversibly immortalized clone, NKNT-3, was selected from the SSR 69 transduction experiments. NKNT-3 cells became immortal without an obvious growth crisis, grew in monolayers in the chemically-defined serum-free medium, CS-C, and doubled in number about every 48 hours. NKNT-3 cells displayed morphological characteristics of liver parenchyma cells such as cytoplasmic granules and large nuclei with a few nucleoli.

NKNT-3 cells expressed SV40T, as assessed by immunofluorescence staining. NKNT-3 cells were not tumorigenic after transplantation into severe combined immunodeficiency (SCID) mice.

To determine whether excision of the transferred oncogene from the NKNT-3 genome could be achieved in the presence of Cre recombinase, NKNT-3 cells were transduced with a replication-deficient recombinant adenovirus (Ad) that expresses the Cre recombinase tagged with a nuclear localization signal(NLS)(Fig. 6). AxCANCre was obtained from Riken Gene Bank (Ibaragi, Japan). It expresses an NLS-tagged Cre recombinase under control of the CAG promoter with viral titers of 3×10⁸ PFU/ml. As expected, transient expression of Cre recombinase triggered a switch in gene expression: Cells became resistant to G418 and no longer expressed SV40T, as assessed by Southern blot, reverse transcription polymerase chain reaction (RT-PCR), and Western blot analyses (FIG. 7A). After a 7 d period of selection with G418, complete elimination of cells expressing SV40T was achieved beyond the limit of detection (FIG. 7A). To ensure that SV40T had been permanently removed from virtually all cells, a thorough examination was performed on a large number of cells, by immunofluorescence with a labeled monoclonal antibody to SV40T, both in vitro, and after intrasplenic transplantation in vivo. After removal of SV40T, NKNT-3 cells were observed to be more differentiated, with nucleus to cytoplasm ratios and number of cytogranules comparable to those of normal primary hepatocytes; they could no longer proliferate. On the basis of these data, a combination of infection with AxCANCre at multiplicity of infection (MOI)10 followed by selection with G418 (500 μg/ml) for 7 d was adopted as the standard protocol for further studies.

Northern Analyses: Patterns of expression of key liver enzymes were determined by Northern analysis. Bil-UDT, GS, and GST-p mRNAs were expressed in NKNT-3 cells before reversal of immortalization but their levels increased substantially after excision of the SV40T gene. Furthermore, albumin and HBCF-X mRNAs could only be detected after removal of the SV40T gene (FIG. 7C). This was not observed with a control Ad expressing LacZ.

### NKNT-3 cells in a Model of Acute Liver Failure

NKNT-3 cells were evaluated after their transplantation in a rat model of acute liver failure (ALF). NKNT-3 cells, before or after recombination, were transplanted into the spleen of rats with ALF induced by 90% hepatectomy. This surgical procedure provided superior reproducibility when compared with ALF induced by hepatotoxins or liver ischemia (Demetriou, Science 233: 1190, 1986; Arkadopoulos et al., Hepatology 28: 1365, 1998; Schumacher et al., Hepatology 24: 337, 1996; Nakamura et al., Transplantation 63: 1541, 1997). In this model, 100% of rats having undergone 90% hepatectomy die of ALF within 3 d. Ninety percent of hepatectomized rats having undergone transplantation of NKNT-3 or reverted NKNT-3 cells showed significant improvement in all parameters (FIG. 8, A, B, and C)and significantly better survival rate when compared with G1 rats (FIG. 8D). G3 rats which received the reverted NKNT-3 cells had higher survival rates compared with G2 rats, which received the nonreverted cells, but this was not statistically significant (FIG. 8D). In another cohort study, rats that survived were killed 4 d after transplantation for anatomical and histopathological examinations. In the surviving rats, the remnant caudate lobe of the liver was markedly enlarged, a characteristic sign of ongoing liver regeneration. NKNT-3 cells and reverted NKNT-3 cells were found in the spleen on histological sections. Islands of splenic “hepatization”were also apparent with reverted NKNT-3 cells, and extracellular bile accumulation was observed around transplanted cells because of the lack of drainage system in the spleen.

### DISCUSSION

A candidate hepatocyte cell line must meet at least one or more functional and/or biochemical requirements to alleviate acute liver failure: it must do or more, and preferably, all of the following: (i) reduce bilirubinemia and jaundice, (ii) improve hepatic encephalopathy due to hyperammonemia (see Example 3 below),(iii) eliminate other toxic substances, and (iv) produce clotting factors (Schumacher et al, supra; Nakamura et al, supra). Decreasing hyperammonemia seems especially important to prevent the development of hepatic encephalopathy and brain death (Strom et al, Transplantation 63: 559, 1997; Fox et al., N. Engl. J. Med 338: 1422, 1998; Chowdhury et al., Pediatrics 102: 647, 1998; Strom et al., Semin. Liver Dis. 19: 39,1999). As glutamine synthetase (GS) is the main contributing enzyme responsible for ammonia clearance, high expression of GS appears essential (Gebhardt et al., Histochemistry 92: 337, 1989).

As is evident from the results, NKNT-3 successfully meets all of these functional criteria for a cell line to alleviate acute liver failure.

These findings demonstrate the feasibility of controlling the expansion of primary human hepatocytes by Cre/Lox-based reversible immortalization with adequate preservation of metabolic functions, which become in fact further enhanced after removal of the transferred oncogene. These data also indicate that transplanted NKNT-3, whether reverted or not, survive in the spleen of rats and provide adequate metabolic support during ALF until the native liver recovers.

Intrasplenic HTX of 5×10⁷ reversibly immortalized human hepatocytes, equivalent to about 5% of the total number of hepatocytes per adult rat, was able to protect animals from ALF. A remaining question is how can a relatively small number of hepatocytes enable hepatectomized rats to recover from ALF?

Although the exact mechanism may remain unknown, considering that all rats submitted to 90% hepatectomy without transplantation (Gl rats) died within 36 hours after surgery and that most rats survive after 80% hepatectomy with no special treatment, 10% additional liver mass equivalent appears sufficient to bridge life until spontaneous regeneration of the liver occurs. One can therefore surmise that intrasplenic injection of 5×10⁷ cells ( 5% liver mass equivalent) may indeed suffice to ensure short-term survival.

Three types of safeguards make it unlikely that transplanted and reverted NKNT-3 cells would expose patients to any oncogenic risk: (i)efficient elimination of the transferred oncogene by site-specific recombination followed by differential selection, (ii) allogeneic transplantation requiring temporary immunosuppression, and (iii) incorporation of a gene (Hygro-TK) “suicide” in the presence of ganciclovir.

For clinical applications in humans, preintegration in the NKNT-3 cell line of an inducible Cre expression cassette (e.g., by tetracycline-mediated induction) (Gossen et al., Science 268: 1766, 1995) may allow considerable expansion of the cell population before reversion without the need for adenoviral superinfection. In addition, replacing the NeoR gene with that encoding the green fluorescent protein would allow efficient and rapid isolation of cells having undergone Cre-mediated recombination (Pawliuk et al., Nature Med 5: 768, 1999).

As alternatives to direct intrasplenic implantation, NKNT-3 cells may be placed in immunoprotective microcapsules or used to generate or to populate bioartifical liver support . These and other means for providing the biofunctional, and or enzymatic properties of the liver cells are contemplated herein. This approach may prove to be a valuable therapeutic strategy to surmount the problem of organ shortage that currently limits the use of liver or hepatocyte transplantation. In the future, reversible immortalization procedures may be extended to other somatic cells with potential applications in various medical conditions.

The present invention is not limited to the embodiments described above, but is capable of variation and modification without departure from the scope of the appended claims.

We claim:

1. A method of making a population of functional hepatocytes for transplantation into a patient, which comprises:
a) providing a sample of primary hepatocytes;
b) immortalizing the hepatocytes by transforming the hepatocytes with a DNA construct comprising a removable DNA segment containing an oncogene, thereby producing immortalized hepatocytes;
c) growing the immortalized hepatocytes; and
d) removing the oncogene from the immortalized hepatocytes, the removal resulting in the production of the population of functional hepatocytes for transplantation into the patient.

2. The method of claim 1, wherein the hepatocytes are obtained from a human donor.

3. The method of claim 1, wherein the oncogene is made removable by flanking it with recombinase target sites, and the removing is accomplished by introducing into the immortalized cells a gene that is expressed to produce a recombinase that specifically recognizes the recombinase target sites.

4. The method of claim 3, wherein the recombinase is Cre recombinase and the recombinase target sites are loxP sites.

5. The method of claim 1, wherein the oncogene is a gene encoding SV40 large T antigen.

6. The method of claim 1, wherein the removable DNA segment further contains a suicide gene, which encodes a gene product that enables destruction of the immortalized cells by an exogenous agent if the removable DNA segment is not removed from the cells.

7. The method of claim 6, wherein the suicide gene is a gene encoding herpes simplex virus thymidine kinase, and the cells are destroyed by exposure to gancyclovir if the removable DNA segment is not removed from the cells.

8. A population of functional hepatocytes produced by the method of claim 1.

9. A method of treating a patient for hepatic failure, comprising transplanting into the patient a sufficient quantity of the hepatocytes of claim 8 to provide hepatic function to the patient.

10. A method of making a population of functional hepatocytes for supporting hepatic function of a patient, which comprises:
a) providing a sample of primary hepatocytes;
b) immortalizing the hepatocytes by transforming the hepatocytes with a DNA construct comprising a removable DNA segment containing an oncogene, a selectable marker gene, and a gene encoding herpes simplex virus thymidine kinase, the genes together being flanked on either side by loxP sites;
c) growing the immortalized hepatocytes; and
d) reversing the immortalization of the hepatocytes by removing the DNA segment from the immortalized hepatocytes, the removing being accomplished by introducing into the immortalized hepatocytes a gene encoding Cre recombinase to effect excision of the DNA segment at the loxP sites, the excision resulting in the production of the population of functional hepatocytes for supporting hepatic function of a patient.

11. The method of claim 10, wherein the hepatocytes are obtained from a human donor.

12. The method of claim 10, wherein the oncogene is a gene encoding SV40 large T antigen.

13. The method of claim 10, wherein the selectable marker gene confers hygromycin resistance to cells expressing the gene.

14. A population of functional hepatocytes produced by the method of claim 10.

15. A method of treating a patient for hepatic failure, comprising transplanting into the patient a sufficient quantity of the hepatocytes of claim 14 to provide hepatic function to the patient.

16. An immortalized hepatocyte comprising a primary hepatocyte transformed with a DNA construct comprising two recombinase target sites that flank an oncogene which confers immortalization to the hepatocyte, wherein the immortalization is reversible by excision of the oncogene by cleavage at the recombinase target sites when the target sites are exposed to a recombinase that specifically recognizes the target sites.

17. The immortalized hepatocyte of claim 16, wherein the recombinase target sites are loxP sites and the immortalization is reversible by Cre recombinase cleavage at the loxP sites.

18. The immortalized hepatocyte of claim 16, wherein the DNA construct further comprises a selectable marker gene.

19. The immortalized hepatocyte of claim 16, wherein the DNA construct further comprises a suicide gene, which encodes a gene product that enables destruction of the immortalized hepatocyte by an exogenous agent if the oncogene is not removed from the cells.

20. The immortalized hepatocyte of claim 19, wherein the suicide gene is a gene encoding herpes simplex virus thymidine kinase, and the exogenous agent is gancyclovir.

21. The immortalized hepatocyte of claim 16, wherein the hepatocyte is obtained from a human donor.

22. A cell line comprising a population of the immortalized hepatocyte of claim 21, which is cell line NKNT-3.

23. The immortalized hepatocyte of claim 20, wherein the hepatocyte is obtained from a rat donor.

24. A cell line comprising a population of the immortalized hepatocyte of claim 23, which is cell line C8-B.

25. A reverse-immortalized hepatocyte that is functional upon transplantation into a patient, produced by exposing the DNA construct within the immortalized hepatocyte of claim 16 to a recombinase that excises the oncogene by cleavage at the recombinase target sites.

26. A method of treating a patient for hepatic failure, comprising transplanting into the patient a sufficient quantity of the reverse-immortalized hepatocytes of claim 25 to provide hepatic function to the patient.
